(19) United States
(12) Patent Application Publication
(10) Pub. No.: US 2023/0089484 A1
(43) Pub. Date: Mar. 23, 2023

(54) COMPOSITIONS FOR THE TREATMENT OF ANGIOLIPOMA

(71) Applicant: Raziel Therapeutics Ltd., Rehovot (IL)

(72) Inventors: Eran BLAUGRUND, Rehovot (IL); Racheli GUETA, Rehovot (IL); Alon BLOOMENFELD, Rehovot (IL)

(21) Appl. No.: 17/795,204

(22) PCT Filed: Jan. 27, 2021
(86) PCT No.: PCT/IB2021/000035

§ 371 Date: Jul. 25, 2022

Related U.S. Application Data
(60) Provisional application No. 62/966,577, filed on Jan. 28, 2020.

(51) Int. Cl. A61K 31/403; A61K 9/00; A61P 35/00

(52) U.S. Cl. CPC A61K 31/403; A61K 9/0019; A61P 35/00

(57) ABSTRACT

The invention provides carbazole derivatives for the treatment of angiolipoma in tissues and organs, and related symptoms, and conditions thereof.

### CROSS-REFERENCE

This application claims the benefit of U.S. Provisional Application No. 62/966,577, filed Jan. 28, 2020, which is incorporated herein by reference.

### BACKGROUND

Angiolipomas, subcutaneous tumors of the extremities and trunk, account for approximately 10% of fatty tumors. They are often multiple, with the first tumors appearing just after puberty. A family history is found in about 10% of cases, but no pattern of inheritance has been proposed. In rare cases, a history of previous trauma to the site or the therapeutic use of protease inhibitors have been implicated. In one case, numerous angiolipomas appeared in a young adult male with familial angio-lipomatosis upon starting anabolic steroids. Mild pain or discomfort is often noted when pressure is applied or the lesions are moved, and the pain appears to be related to the vascularity of the lesions.

Subcutaneous angiolipomas have a normal karyotype, setting them apart from most other fatty tumors, including lipomas. For this reason, they have been regarded as a hamartoma of blood vessels and fat, rather than as a true fatty tumor. Macroscopically, they are yellow, firm, circumscribed tumors, rang in diameter from 1 to 4 cm. They may have a yellow-red appearance on the cut surface, reflecting the degree of vascularity. Subcutaneous angiolipomas must be distinguished from the infiltrating angiolipoma, which is a solitary lesion of the deep soft tissues.

In contrast to lipomas that contain only fat tissue, angiolipomas have a thin fibrous capsule with incomplete fibrous septa extending into the lesion, dividing it into lobules of different size. They are composed of variable proportions of fatty tissue and blood vessels. The fat cells are mature, with a single vacuole and an eccentric nucleus. The vascular component, which comprises 5-50% or more of the tumor, consists of groups of capillaries and occasional vessels of larger caliber. Only sparse fat cells are present in the lesion reported as a cellular angiolipoma, which are composed almost entirely of small vessels. In most cases, angiolipomas display prominent pericytes around the vessels. Erythrocytes are present within the lumen, and scattered fibrin thrombi are easily found. In one report, hemorrhagic infarction of the fat was present in association with numerous fibrin thrombi and disseminated intravascular coagulation. Thus, angiolipomas seem to be less prone to fat reducing agents due to the fibrotic tissue they contain. While fat reducing agents can eliminate fat from angiolipomas, they cannot interact with the fibrous cap that lines the borders of angiolipomas and are therefore expected to be ineffective at reducing angiolipoma size.

Angiolipomas can cause significant pain by pressing on adjacent nerves. Treatment of angiolipomas is usually by surgery or, in some cases, by injection of corticosteroids into the cell mass. Hence, there remains a need for the development of further agents for the treatment of angiolipomas.

### SUMMARY

In one aspect, provided herein is a pharmaceutical composition comprising a compound of Formula (I):

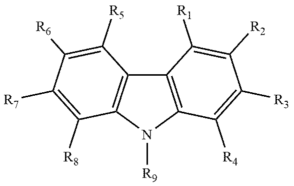

or a pharmaceutically acceptable salt thereof, wherein
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, -NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, -NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋₅alkynyl, C₁-sheteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
for use in the treatment of angiolipoma and any symptoms or conditions associated therewith.

In some embodiments, R₉ is C₁-C₉ alkyl substituted with at least one quaternary amino group.

In some embodiments, the at least one ammonium group is of Formula (V):

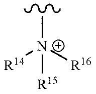

wherein each of R¹⁴, R¹⁵, and R¹⁶ is independently selected from C₁₋₉alkyl, C₂₋₉alkenyl, and C₂₋₉₋ alkynyl. In some embodiments, each of R¹⁴, R¹⁵, and R¹⁶ is independently C₁₋₉alkyl.

In some embodiments, at least one of R¹, R², R³, and R⁴ is halogen. In some embodiments, at least one of R⁵, R⁶, R⁷, and R⁸ is halogen. In some embodiments, at least one of R1, R², R³, and R⁴ is halogen and at least one of R⁵, R⁶, R⁷, and R⁸ is halogen. In some embodiments, the halogen is bromo.

In some embodiments, at least one of R¹, R², R³, and R⁴ is OH. In some embodiments, at least one of R⁵, R⁶, R⁷, and R⁸ is OH.

In some embodiments, at least one of R¹, R², R³, and R⁴ is nitro and at least one of R⁵, R⁶, R⁷, and R⁸ is nitro.

In some embodiments, the compound of Formula (I) is selected from:
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, and
5-(3,6-dibromo-9H-carbazol-9-yl)-N,N,N-trimethylpentan-1-aminium.

In some embodiments, the pharmaceutical composition comprises less than about 50% water by weight. In some embodiments, the pharmaceutical composition comprises less than about 30% water by weight. In some embodiments, the pharmaceutical composition comprises less than about 10% water by weight. In some embodiments, the pharmaceutical composition comprises from about 0% to about 30% water by weight.

In some embodiments, the pharmaceutical composition comprises at least about 0.1% by weight of the compound of Formula (I). In some embodiments, the pharmaceutical composition comprises from about 0.1% to about 10% by weight of the compound of Formula (I). In some embodiments, the pharmaceutical composition comprises from about 1% to about 5% by weight of the compound of Formula (I).

In some embodiments, the pharmaceutical composition further comprises at least one additional therapeutic agent.

In some embodiments, the pharmaceutical composition is formulated for parenteral administration. In some embodiments, the pharmaceutical composition is formulated for subcutaneous injection directly into an angiolipoma. In some embodiments, the pharmaceutical composition is formulated for subcutaneous injection directly into an angiolipoma at a dosage of from about 0.05 to about 0.1 mL per angiolipoma. In some embodiments, the pharmaceutical composition is formulated for subcutaneous injection directly into an angiolipoma at a dosage of from about 0.1 to about 0.4 mL per angiolipoma. In some embodiments, the pharmaceutical composition is formulated for subcutaneous injection directly into an angiolipoma at a dosage of from about 0.4 to about 1 mL per angiolipoma. In some embodiments, the pharmaceutical composition is formulated for subcutaneous injection directly into an angiolipoma at a dosage of from about 1 to about 2 mL per angiolipoma.

Provided herein, in another aspect, is a kit comprising the pharmaceutical composition provided herein, means for administration of the pharmaceutical composition, and instructions for use thereof.

In some embodiments, the kit further comprises at least one additional therapeutic agent.

Provided herein, in another aspect, is a method of treating angiolipoma in a subject in need thereof, comprising administering to the subject a pharmaceutical composition comprising a compound of Formula (I):

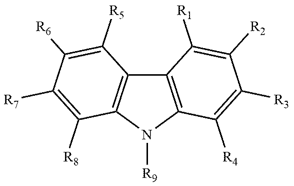

or a pharmaceutically acceptable salt thereof, wherein:
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR13N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋₅alkynyl, C₁-sheteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁-sheteroalkyl, C₁₋₅haloalkyl, and C₃₋₆cycloalkyl.

In some embodiments, the pharmaceutical composition is administered parenterally. In some embodiments, the pharmaceutical composition is administered subcutaneously. In some embodiments, the pharmaceutical composition is subcutaneously injected directly into an angiolipoma. In some embodiments, the pharmaceutical composition is subcutaneously injected directly into an angiolipoma at a dosage of from about 0.05 to about 0.1 mL per angiolipoma. In some embodiments, the pharmaceutical composition is subcutaneously injected directly into an angiolipoma at a dosage of from about 0.1 to about 0.4 mL per angiolipoma. In some embodiments, the pharmaceutical composition is subcutaneously injected directly into an angiolipoma at a dosage of from about 0.4 to about 1 mL per angiolipoma. In some embodiments, the pharmaceutical composition is subcutaneously injected directly into an angiolipoma at a dosage of from about 1 to about 2 mL per angiolipoma.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### DETAILED DESCRIPTION OF THE INVENTION

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x—y}alkylene—refers to a substituted or unsubstituted alkylene chain with from x to y carbons in the alkylene chain. For example —C_{1—6}alkylene— may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized, and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Aryl” refers to an aromatic ring wherein each of the atoms forming the ring is a carbon atom. Aryl groups can be optionally substituted. Examples of aryl groups include, but are not limited to, phenyl and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-”(such as in “aralkyl”) is meant to include aryl radicals that are optionally substituted.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl is optionally substituted by one or more substituents such as those substituents described herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical may be partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (-) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)— or (S)—. The present chemical entities, pharmaceutical compositions and methods are meant to include all such possible stereoisomers, including racemic mixtures, optically pure forms, mixtures of diastereomers and intermediate mixtures. Optically active (R)— and (S)—isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z— or E— form (or cis- or trans- form). Furthermore, some chemical entities may exist in various tautomeric forms. Unless otherwise specified, chemical entities described herein are intended to include all Z—, E— and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E- forms (or cis- and trans- forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

### Compounds

Provided herein, in one aspect, is a compound of Formula (I):

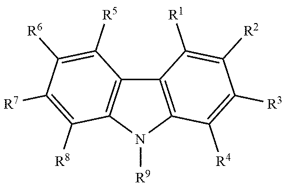

or a pharmaceutically acceptable salt thereof, wherein:
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋₅alkynyl, C₁-sheteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁-sheteroalkyl, C₁₋₅haloalkyl, and C₃₋₆cycloalkyl.

In some embodiments, each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁-salkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O). In some embodiments, each of R¹, R², R³, R⁴, R⁵, R⁶, R⁷, and R⁸ is independently selected from H, halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, and —NR¹³S(═O)₂NR¹¹R¹². In some embodiments, each of R¹, R², R³, R⁴, R⁵, R⁶, R⁷, and R⁸ is independently selected from H, halogen, —CN, —NO₂, —OR¹⁰, —NR¹¹R¹², —C(═O)R¹⁰, —C(═O)OR¹⁰, and C₁₋₅alkyl; wherein each alkyl is independently optionally substituted with one or more substituents selected from halogen, —CN, —NO₂, —OR¹⁰, —S(═O)₂R¹⁰, —NR¹¹R¹², —C(—O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, and —NR¹³S(═O)₂NR¹¹R¹². In some embodiments, each of R¹, R², R³, R⁴, R⁵, R⁶, R⁷, and R⁸ is independently selected from H, halogen, —CN, —NO₂, —OR¹⁰, and —NR¹¹R¹².

In some embodiments, at least one of R¹, R², R³, and R⁴ is halogen. In some embodiments, R¹ is halogen. In some embodiments, R² is halogen. In some embodiments, R³ is halogen. In some embodiments, R⁴ is halogen. In some embodiments, at least one of R⁵, R⁶, R⁷, and R⁸ is halogen. In some embodiments, R⁵ is halogen. In some embodiments, R⁶ is halogen. In some embodiments, R⁷ is halogen. In some embodiments, R⁸ is halogen. In some embodiments, at least one of R¹, R², R³, and R⁴ is halogen and at least one of R⁵, R⁶, R⁷, and R⁸ is halogen. In some embodiments, R¹ is halogen and R⁵ is halogen. In some embodiments, R¹ is halogen and R⁶ is halogen. In some embodiments, R¹ is halogen and R⁷ is halogen. In some embodiments, R¹ is halogen and R⁸ is halogen. In some embodiments, R² is halogen and R⁵ is halogen. In some embodiments, R² is halogen and R⁶ is halogen. In some embodiments, R² is halogen and R⁷ is halogen. In some embodiments, R² is halogen and R⁸ is halogen. In some embodiments, R³ is halogen and R⁵ is halogen. In some embodiments, R³ is halogen and R⁶ is halogen. In some embodiments, R³ is halogen and R⁷ is halogen. In some embodiments, R³ is halogen and R⁸ is halogen. In some embodiments, R⁴ is halogen and R⁵ is halogen. In some embodiments, R⁴ is halogen and R⁶ is halogen. In some embodiments, R⁴ is halogen and R⁷ is halogen. In some embodiments, R⁴ is halogen and R⁸ is halogen. In some embodiments, the halogen is bromo. In some embodiments, the halogen is chloro. In some embodiments, the halogen is fluoro.

In some embodiments, at least one of R¹, R², R³, and R⁴ is OH. In some embodiments, R¹ is OH. In some embodiments, R² is OH. In some embodiments, R³ is OH. In some embodiments, R⁴ is OH. In some embodiments, at least one of R⁵, R⁶, R⁷, and R⁸ is OH. In some embodiments, R⁵ is OH. In some embodiments, R⁶ is OH. In some embodiments, R⁷ is OH. In some embodiments, R⁸ is OH.

In some embodiments, at least one of R¹, R², R³, and R⁴ is nitro and at least one of R⁵, R⁶, R⁷, and R⁸ is nitro. In some embodiments, R¹ is nitro and R⁵ is nitro. In some embodiments, R¹ is nitro and R⁶ is nitro. In some embodiments, R¹ is nitro and R⁷ is nitro. In some embodiments, R¹ is nitro and R⁸ is nitro. In some embodiments, R² is halogen and R⁵ is nitro. In some embodiments, R² is nitro and R⁶ is nitro. In some embodiments, R² is halogen and R⁷ is nitro. In some embodiments, R² is nitro and R⁸ is nitro. In some embodiments, R³ is halogen and R⁵ is nitro. In some embodiments, R³ is nitro and R⁶ is nitro. In some embodiments, R³ is halogen and R⁷ is nitro. In some embodiments, R³ is nitro and R⁸ is nitro. In some embodiments, R⁴ is halogen and R⁵ is nitro. In some embodiments, R⁴ is nitro and R⁶ is nitro. In some embodiments, R⁴ is halogen and R⁷ is nitro. In some embodiments, R⁴ is nitro and R⁸ is nitro.

In some embodiments, R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group. In some embodiments, R⁹ is C₂₋₉alkenyl substituted with at least one phosphonium group. In some embodiments, R⁹ is C₂₋₉alkenyl substituted with at least one quaternary amino group. In some embodiments, R⁹ is C₂₋₉alkynyl substituted with at least one phosphonium group. In some embodiments, R⁹ is C₂₋₉alkynyl substituted with at least one quaternary amino group. In some embodiments, R⁹ is a 3- to 10-membered heterocycloalkyl. In some embodiments, R⁹ is piperazinyl. In some embodiments, R⁹ is pyridinyl. In some embodiments, R⁹ is piperidinyl. In some embodiments, R⁹ is morpholinyl. In some embodiments, R⁹ is thiomorpholinyl. In some embodiments, R⁹ is C₁₋₉alkyl substituted with at least one phosphonium group. In some embodiments, R⁹ is C₁₋₉alkyl substituted with at least one quaternary amino group. In some embodiments, R⁹ is propyl substituted with at least one quaternary amino group. In some embodiments, R⁹ is pentyl substituted with at least one quaternary amino group.

In some embodiments, the at least one quaternary amino group is of Formula (V):

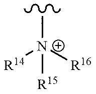

wherein each of R¹⁴, R¹⁵, and R¹⁶ is independently selected from C₁₋₉alkyl, C₂₋₉alkenyl, and C₂₋₉₋ alkynyl. In some embodiments, each of R¹⁴, R¹⁵, and R¹⁶ is independently C₂₋₉alkenyl. In some embodiments, each of R¹⁴, R¹⁵, and R¹⁶ is independently C₂₋₉alkynyl. In some embodiments, each of R¹⁴, R¹⁵, and R¹⁶ is independently C₁₋₉alkyl. In some embodiments, each of R¹⁴, R¹⁵, and R¹⁶ is methyl.

In some embodiments, the at least one phosphonium group is of Formula (VI):

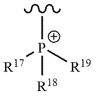

wherein each of R¹⁷, R¹⁸, and R¹⁹ is independently selected from C₁₋₉alkyl, C₂₋₉alkenyl, and C₂₋₉₋ alkynyl. In some embodiments, each of R¹⁷, R¹⁸, and R¹⁹ is independently C₂₋₉alkenyl. In some embodiments, each of R¹⁷, R¹⁸, and R¹⁹ is independently C₂₋₉alkynyl. In some embodiments, each of R¹⁷ , R¹⁸, and R¹⁹ is independently C₁₋₉alkyl. In some embodiments, each of R¹⁷ , R¹⁸, and R¹⁹ is methyl.

In some embodiments, each R¹⁰ is independently selected from H, C₁₋₅alkyl, C₂₋₅alkenyl, C₂-₅alkynyl, C₁₋₅heteroalkyl, C₁₋₅haloalkyl, and C₃₋₆cycloalkyl. In some embodiments, each R¹⁰ is independently selected from H, C₁₋₅alkyl, C₁₋₅heteroalkyl, C₁₋₅haloalkyl, and C₃₋₆cycloalkyl. In some embodiments, each R¹⁰ is independently selected from H, C₁₋₅alkyl, and C₃₋₆cycloalkyl. In some embodiments, each R¹⁰ is H. In some embodiments, each R¹⁰ is independently C₁₋₅alkyl. In some embodiments, each R¹⁰ is independently C₂₋₅alkenyl. In some embodiments, each R¹⁰ is independently C₂₋₅alkynyl. In some embodiments, each R¹⁰ is independently C₁₋₅heteroalkyl. In some embodiments, each R¹⁰ is independently C₁₋₅haloalkyl. In some embodiments, each R¹⁰ is independently C₃₋ ₆cycloalkyl.

In some embodiments, each R¹¹ and R¹² is independently selected from H, C₁₋₅alkyl, C₂₋₅₋ alkenyl, C₂₋₅alkynyl, C₁₋₅heteroalkyl, C₁₋₅haloalkyl, and C₃₋₆cycloalkyl; or an R¹² and an R¹³ may be taken together along with the nitrogen atom to which they are attached to form a 3- to 10-membered heterocycloalkyl. In some embodiments, each R¹¹ and R¹² is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, each R¹¹ and R¹² is independently selected from H, C₁₋₅alkyl, C₁₋₅heteroalkyl, C₁₋₅haloalkyl, and C₃₋ ₆cycloalkyl. In some embodiments, each R¹¹ and R¹² is independently selected from H, C₁₋₅alkyl, and C₃₋₆cycloalkyl. In some embodiments, each R¹¹ and R¹² is H. In some embodiments, each R¹¹ and R¹² is independently C₁₋₅alkyl. In some embodiments, each R¹¹ and R¹² is independently C₂₋₅alkenyl. In some embodiments, each R¹¹ and R¹² is independently C₂₋₅alkynyl. In some embodiments, each R¹¹ and R¹² is independently C₁₋₅heteroalkyl. In some embodiments, each R¹¹ and R¹² is independently C₁₋ shaloalkyl. In some embodiments, each R¹¹ and R¹² is independently C₃₋₆cycloalkyl. In some embodiments, an R¹² and an R¹³ are taken together along with the nitrogen atom to which they are attached to form a 3 - to 10-membered heterocycloalkyl.

In some embodiments, each R¹³ is independently selected from H, C₁₋₅alkyl, C₂₋₅alkenyl, C₂-₅alkynyl, C₁₋₅heteroalkyl, C₁₋₅haloalkyl, and C₃₋₆cycloalkyl. In some embodiments, each R¹³ is independently selected from H, C₁₋₅alkyl, C₁₋₅heteroalkyl, C₁₋₅haloalkyl, and C₃₋₆cycloalkyl. In some embodiments, each R¹³ is independently selected from H, C₁₋₅alkyl, and C₃₋₆cycloalkyl. In some embodiments, each R¹³ is H. In some embodiments, each R¹³ is independently C₁₋₅alkyl. In some embodiments, each R¹³ is independently C₂₋₅alkenyl. In some embodiments, each R¹³ is independently C₂₋₅alkynyl. In some embodiments, each R¹³ is independently C₁₋₅heteroalkyl. In some embodiments, each R¹³ is independently C₁₋₅haloalkyl. In some embodiments, each R¹³ is independently C₃₋ ₆cycloalkyl.

In some embodiments, the compound of Formula (I) is selected from:
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, and
5-(3,6-dibromo-9H-carbazol-9-yl)-N,N,N-trimethylpentan-1-aminium.

In some embodiments, the compound of Formula (I) is 3-(3,6-dibromo-9H-carbazol-9-yl)-N,N,N-trimethylpropan-1-aminium. In some embodiments, the compound of Formula (I) is 5-(9H-carbazol-9-yl)-N,N,N-trimethylpentan-1-aminium. In some embodiments, the compound of Formula (I) is 5-(2-hydroxy-9H-carbazol-9-yl)-N,N,N-trimethylpentan-1-aminium. In some embodiments, the compound of Formula (I) is 5-(3,6-dibromo-9H-carbazol-9-yl)-N,N,N-trimethylpentan-1-aminium.

In some embodiments, the compound of Formula (I) is represented by the structure:

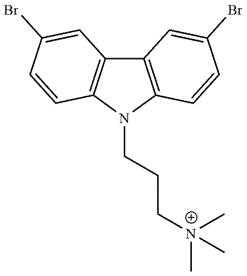 [SMILES: C[N+](C)(C)CCCn1c2ccc(Br)cc2c2cc(Br)ccc21]

. In some embodiments, the compound of Formula (I) is represented by the structure:

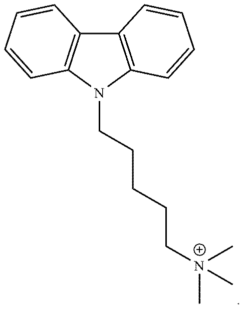

In some embodiments, the compound of Formula (I) is represented by the structure:

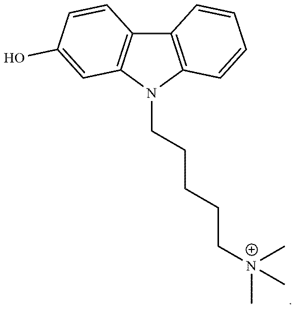

. In some embodiments, the compound of Formula (I) is represented by the structure:

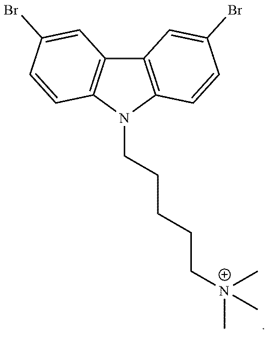

### Pharmaceutical Compositions

A composition of the present disclosure may be formulated in any suitable pharmaceutical formulation. A pharmaceutical composition of the present disclosure typically contains an active ingredient (e.g., a compound of Formula (I), or a pharmaceutically acceptable salt and/or coordination complex thereof), and one or more pharmaceutically acceptable excipients or carriers, including but not limited to: inert solid diluents and fillers, diluents, sterile aqueous solution and various organic solvents, permeation enhancers, solubilizers, and adjuvants. A composition of the present disclosure may be formulated in any suitable pharmaceutical formulation.

Pharmaceutical compositions may be provided in any suitable form, which may depend on the route of administration. In some embodiments, the pharmaceutical composition disclosed herein can be formulated in dosage form for administration to a subject. In some embodiments, the pharmaceutical composition is formulated for parenteral, topical, transdermal, buccal, sublingual, subcutaneous, intramuscular, intravenous, intratumoral, and/or intraperitoneal administration. In some embodiments, the pharmaceutical composition can be formulated as a unit dosage.

The amount of each compound administered will be dependent on the mammal being treated, the severity of the disorder or condition, the rate of administration, the disposition of the compound and the discretion of the prescribing physician. However, an effective dosage may be in the range of about 0.001 to about 100 mg per kg body weight per day, in single or divided doses. In some instances, dosage levels below the lower limit of the aforesaid range may be more than adequate, while in other cases still larger doses may be employed without causing any harmful side effect, e.g., by dividing such larger doses into several small doses for administration throughout the day. In some embodiments, an effective dosage may be provided in pulsed dosing (i.e., administration of the compound in consecutive days, followed by consecutive days of rest from administration).

In some embodiments, the composition is provided in one or more unit doses. For example, the composition can be administered in 1, 2, 3, 4, 5, 6, 7, 14, 30, 60, or more doses. Such amount can be administered each day, for example in individual doses administered once, twice, or three or more times a day. However, dosages stated herein on a per day basis should not be construed to require administration of the daily dose each and every day. For example, if one of the agents is provided in a suitably slow-release form, two or more daily dosage amounts can be administered at a lower frequency, e.g., as a depot injection administered every second day to once a month or even longer. Most typically and conveniently for the subject, a pharmaceutical composition comprising a compound of Formula (I) can be administered once a day, for example in the morning, in the evening or during the day.

The unit doses can be administered simultaneously or sequentially. The composition can be administered for an extended treatment period. Illustratively, the treatment period can be at least about one month, for example at least about 3 months, at least about 6 months or at least about 1 year. In some cases, administration can continue for substantially the remainder of the life of the subject.

In some embodiments, the pharmaceutical composition comprising the compound of Formula (I) can be administered as part of a therapeutic regimen that comprises administering one or more second agents (e.g. 1, 2, 3, 4, 5, or more second agents), either simultaneously or sequentially with the pharmaceutical composition comprising the compound of Formula (I). When administered sequentially, the pharmaceutical composition comprising the compound of Formula (I) may be administered before or after the one or more second agents. When administered simultaneously, the pharmaceutical composition comprising the compound of Formula (I) and the one or more second agents may be administered by the same route (e.g. injections to the same location), by a different route (e.g. a tablet taken orally while receiving an intravenous infusion), or as part of the same combination (e.g. a solution comprising the pharmaceutical composition comprising the compound of Formula (I) and one or more second agents).

A combination treatment according to the invention may be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the agent selected, the route of administration, the form in which the compound is administered, the subject to be treated, the body weight of the subject to be treated, and the preference and experience of the attending physician.

In some embodiments, the pharmaceutical composition comprises one or more surfactants. Surfactants which can be used to form pharmaceutical composition and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono-and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group of vegetable oils, hydrogenated vegetable oils, and triglycerides.

In one embodiment, the composition may include a solubilizer to ensure good solubilization and/or dissolution of the compound of the present disclosure and to minimize precipitation of the compound of the present disclosure. This can be especially important for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. If present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Provided herein, in one aspect, is a pharmaceutical composition comprising a compound of Formula (I):

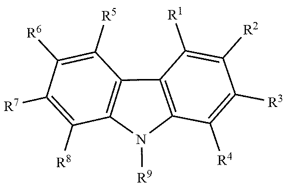

or a pharmaceutically acceptable salt thereof, wherein:
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋₅alkynyl, C₁-sheteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁-sheteroalkyl, C₁₋₅haloalkyl, and C₃₋₆cycloalkyl.

In some embodiments, the pharmaceutical composition comprises less than about 50% water by weight. In some embodiments, the pharmaceutical composition comprises less than about 30% water by weight. In some embodiments, the pharmaceutical composition comprises less than about 10% water by weight. In some embodiments, the pharmaceutical composition comprises from about 0% to about 30% water by weight. In some embodiments, the pharmaceutical composition comprises from about 10% to about 30% water by weight. In some embodiments, the pharmaceutical composition comprises from about 15% to about 30% water by weight. In some embodiments, the pharmaceutical composition comprises from about 15% to about 25% water by weight. In some embodiments, the pharmaceutical composition comprises from about 20% to about 30% water by weight. In some embodiments, the pharmaceutical composition comprises from about 23% to about 27% water by weight. In some embodiments, the pharmaceutical composition comprises from about 24% to about 26% water by weight. In some embodiments, the pharmaceutical composition comprises about 0% water by weight. In some embodiments, the pharmaceutical composition comprises about 1% water by weight. In some embodiments, the pharmaceutical composition comprises about 2% water by weight. In some embodiments, the pharmaceutical composition comprises about 3% water by weight. In some embodiments, the pharmaceutical composition comprises about 4% water by weight. In some embodiments, the pharmaceutical composition comprises about 5% water by weight. In some embodiments, the pharmaceutical composition comprises about 6% water by weight. In some embodiments, the pharmaceutical composition comprises about 7% water by weight. In some embodiments, the pharmaceutical composition comprises about 8% water by weight. In some embodiments, the pharmaceutical composition comprises about 9% water by weight. In some embodiments, the pharmaceutical composition comprises about 10% water by weight. In some embodiments, the pharmaceutical composition comprises about 11% water by weight. In some embodiments, the pharmaceutical composition comprises about 12% water by weight. In some embodiments, the pharmaceutical composition comprises about 13% water by weight. In some embodiments, the pharmaceutical composition comprises about 14% water by weight. In some embodiments, the pharmaceutical composition comprises about 15% water by weight. In some embodiments, the pharmaceutical composition comprises about 16% water by weight. In some embodiments, the pharmaceutical composition comprises about 17% water by weight. In some embodiments, the pharmaceutical composition comprises about 18% water by weight. In some embodiments, the pharmaceutical composition comprises about 19% water by weight. In some embodiments, the pharmaceutical composition comprises about 20% water by weight. In some embodiments, the pharmaceutical composition comprises about 21% water by weight. In some embodiments, the pharmaceutical composition comprises about 22% water by weight. In some embodiments, the pharmaceutical composition comprises about 23% water by weight. In some embodiments, the pharmaceutical composition comprises about 24% water by weight. In some embodiments, the pharmaceutical composition comprises about 25% water by weight. In some embodiments, the pharmaceutical composition comprises about 26% water by weight. In some embodiments, the pharmaceutical composition comprises about 27% water by weight. In some embodiments, the pharmaceutical composition comprises about 28% water by weight. In some embodiments, the pharmaceutical composition comprises about 29% water by weight. In some embodiments, the pharmaceutical composition comprises about 30% water by weight.

In some embodiments, the pharmaceutical composition comprises at least about 0.1% by weight of the compound of Formula (I). In some embodiments, the pharmaceutical composition comprises between about 0.1% to about 10% by weight of the compound of Formula (I). In some embodiments, the pharmaceutical composition comprises between about 1% to about 5% by weight of the compound of Formula (I). In some embodiments, the pharmaceutical composition comprises about 0.1% by weight of the compound of Formula (I). In some embodiments, the pharmaceutical composition comprises about 0.2% by weight of the compound of Formula (I). In some embodiments, the pharmaceutical composition comprises about 0.3% by weight of the compound of Formula (I). In some embodiments, the pharmaceutical composition comprises about 0.4% by weight of the compound of Formula (I). In some embodiments, the pharmaceutical composition comprises about 0.5% by weight of the compound of Formula (I). In some embodiments, the pharmaceutical composition comprises about 0.6% by weight of the compound of Formula (I). In some embodiments, the pharmaceutical composition comprises about 0.7% by weight of the compound of Formula (I). In some embodiments, the pharmaceutical composition comprises about 0.8% by weight of the compound of Formula (I). In some embodiments, the pharmaceutical composition comprises about 0.9% by weight of the compound of Formula (I). In some embodiments, the pharmaceutical composition comprises about 1% by weight of the compound of Formula (I). In some embodiments, the pharmaceutical composition comprises about 1.5% by weight of the compound of Formula (I). In some embodiments, the pharmaceutical composition comprises about 2% by weight of the compound of Formula (I). In some embodiments, the pharmaceutical composition comprises about 2.5% by weight of the compound of Formula (I). In some embodiments, the pharmaceutical composition comprises about 3% by weight of the compound of Formula (I). In some embodiments, the pharmaceutical composition comprises about 3.5% by weight of the compound of Formula (I). In some embodiments, the pharmaceutical composition comprises about 4% by weight of the compound of Formula (I). In some embodiments, the pharmaceutical composition comprises about 4.5% by weight of the compound of Formula (I). In some embodiments, the pharmaceutical composition comprises about 5% by weight of the compound of Formula (I). In some embodiments, the pharmaceutical composition comprises about 5.5% by weight of the compound of Formula (I). In some embodiments, the pharmaceutical composition comprises about 6% by weight of the compound of Formula (I). In some embodiments, the pharmaceutical composition comprises about 6.5% by weight of the compound of Formula (I). In some embodiments, the pharmaceutical composition comprises about 7% by weight of the compound of Formula (I). In some embodiments, the pharmaceutical composition comprises about 7.5% by weight of the compound of Formula (I). In some embodiments, the pharmaceutical composition comprises about 8% by weight of the compound of Formula (I). In some embodiments, the pharmaceutical composition comprises about 8.5% by weight of the compound of Formula (I). In some embodiments, the pharmaceutical composition comprises about 9% by weight of the compound of Formula (I). In some embodiments, the pharmaceutical composition comprises about 9.5% by weight of the compound of Formula (I). In some embodiments, the pharmaceutical composition comprises about 10% by weight of the compound of Formula (I).

In some embodiments, the pharmaceutical composition further comprises at least one additional active agent. In some embodiments, the additional active agent is a cytotoxic agent.

In some embodiments, the pharmaceutical composition is formulated for parenteral, topical, transdermal, buccal, sublingual, subcutaneous, intramuscular, intravenous, intratumoral, and/or intraperitoneal administration. In some embodiments, the pharmaceutical composition is formulated for parenteral administration. In some embodiments, the pharmaceutical composition is formulated for injection. In some embodiments, the pharmaceutical composition is formulated for intratumoral injection. In some embodiments, the pharmaceutical composition is formulated as an injection, a patch, a cream, a gel, or an ointment.

### Pharmaceutical Compositions for Injection

In some embodiments, the disclosure provides a pharmaceutical composition for injection containing a compound of Formula (I) and a pharmaceutical excipient suitable for injection. Components and amounts of agents in the composition are as described herein.

The forms in which the novel composition of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

The invention also provides kits. The kits may include a pharmaceutical composition comprising a compound of Formula (I) and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Methods of Use

Eliminating or reducing angiolipomas is significantly different than treating regular lipomas, as the former contain fibrous tissue. This fibrous capsule is capable of keeping the angiolipoma firm even when a fat reducing agent is injected. Although angiolipomas are expected to be resistant to fat reducing agents, provided herein is a fat-reducing compound for the treatment of angiolipomas. A single injection of Compound 1 into lipomas containing fibrous capsules is capable of significantly reducing their height and alleviating angiolipoma-associated pain.

In one aspect, provided herein is a pharmaceutical composition comprising a compound of Formula (I):

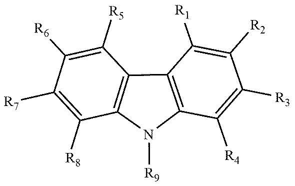

or a pharmaceutically acceptable salt thereof, wherein
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋₅alkynyl, C₁-sheteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁-sheteroalkyl, C₁₋₅haloalkyl, and C₃₋₆cycloalkyl;
   for use in the treatment of angiolipoma and any symptoms or conditions associated therewith.

In some embodiments, the pharmaceutical composition further comprises at least one additional therapeutic agent.

In some embodiments, the pharmaceutical composition is formulated for parenteral administration. In some embodiments, the pharmaceutical composition is formulated for subcutaneous injection directly into an angiolipoma. In some embodiments, the pharmaceutical composition is formulated for subcutaneous injection directly into an angiolipoma at a dosage of from about 0.05 to about 0.1 mL per angiolipoma. In some embodiments, the pharmaceutical composition is formulated for subcutaneous injection directly into an angiolipoma at a dosage of from about 0.1 to about 0.4 mL per angiolipoma. In some embodiments, the pharmaceutical composition is formulated for subcutaneous injection directly into an angiolipoma at a dosage of from about 0.4 to about 1 mL per angiolipoma. In some embodiments, the pharmaceutical composition is formulated for subcutaneous injection directly into an angiolipoma at a dosage of from about 1 to about 2 mL per angiolipoma. In some embodiments, the pharmaceutical composition is formulated for subcutaneous injection directly into an angiolipoma at a dosage of less than about 0.05 mL per angiolipoma. In some embodiments, the pharmaceutical composition is formulated for subcutaneous injection directly into an angiolipoma at a dosage of about 0.05 mL per angiolipoma. In some embodiments, the pharmaceutical composition is formulated for subcutaneous injection directly into an angiolipoma at a dosage of about 0.1 mL per angiolipoma. In some embodiments, the pharmaceutical composition is formulated for subcutaneous injection directly into an angiolipoma at a dosage of about 0.2 mL per angiolipoma. In some embodiments, the pharmaceutical composition is formulated for subcutaneous injection directly into an angiolipoma at a dosage of about 0.3 mL per angiolipoma. In some embodiments, the pharmaceutical composition is formulated for subcutaneous injection directly into an angiolipoma at a dosage of about 0.4 mL per angiolipoma. In some embodiments, the pharmaceutical composition is formulated for subcutaneous injection directly into an angiolipoma at a dosage of about 0.5 mL per angiolipoma. In some embodiments, the pharmaceutical composition is formulated for subcutaneous injection directly into an angiolipoma at a dosage of about 0.6 mL per angiolipoma. In some embodiments, the pharmaceutical composition is formulated for subcutaneous injection directly into an angiolipoma at a dosage of about 0.7 mL per angiolipoma. In some embodiments, the pharmaceutical composition is formulated for subcutaneous injection directly into an angiolipoma at a dosage of about 0.8 mL per angiolipoma. In some embodiments, the pharmaceutical composition is formulated for subcutaneous injection directly into an angiolipoma at a dosage of about 0.9 mL per angiolipoma. In some embodiments, the pharmaceutical composition is formulated for subcutaneous injection directly into an angiolipoma at a dosage of about 1.0 mL per angiolipoma. In some embodiments, the pharmaceutical composition is formulated for subcutaneous injection directly into an angiolipoma at a dosage of about 1.1 mL per angiolipoma. In some embodiments, the pharmaceutical composition is formulated for subcutaneous injection directly into an angiolipoma at a dosage of about 1.2 mL per angiolipoma. In some embodiments, the pharmaceutical composition is formulated for subcutaneous injection directly into an angiolipoma at a dosage of about 1.3 mL per angiolipoma. In some embodiments, the pharmaceutical composition is formulated for subcutaneous injection directly into an angiolipoma at a dosage of about 1.4 mL per angiolipoma. In some embodiments, the pharmaceutical composition is formulated for subcutaneous injection directly into an angiolipoma at a dosage of about 1.5 mL per angiolipoma. In some embodiments, the pharmaceutical composition is formulated for subcutaneous injection directly into an angiolipoma at a dosage of about 1.6 mL per angiolipoma. In some embodiments, the pharmaceutical composition is formulated for subcutaneous injection directly into an angiolipoma at a dosage of about 1.7 mL per angiolipoma. In some embodiments, the pharmaceutical composition is formulated for subcutaneous injection directly into an angiolipoma at a dosage of about 1.8 mL per angiolipoma. In some embodiments, the pharmaceutical composition is formulated for subcutaneous injection directly into an angiolipoma at a dosage of about 1.9 mL per angiolipoma. In some embodiments, the pharmaceutical composition is formulated for subcutaneous injection directly into an angiolipoma at a dosage of about 2.0 mL per angiolipoma. In some embodiments, the pharmaceutical composition is formulated for subcutaneous injection directly into an angiolipoma at a dosage of greater than about 2.0 mL per angiolipoma.

Provided herein, in another aspect, is a method of treating angiolipoma in a subject in need thereof, comprising administering to the subject a pharmaceutical composition comprising a compound of Formula (I):

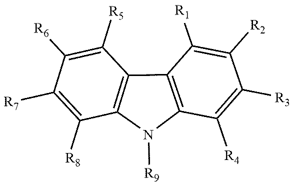

or a pharmaceutically acceptable salt thereof, wherein:
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)zR^{l°}, —NR¹¹R¹², —C(═O)NR¹¹R¹², —S(═O)NR¹¹R¹², —S(═O)₂NR¹¹R ¹², —C(═O)R¹⁰, —C(═O)OR¹⁰, —NR¹³C(═O)R¹⁰, —NR¹³C(═O)NR¹¹R¹², —NR¹³S(═O)₂R¹⁰, _{—}NR¹³S(═O)₂NR¹¹R¹², _{—}C(═S)R¹⁰, _{—}N(═O), —SN(═O), —NR13N(═O), _{—}ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂-₅alkynyl, C₁₋ sheteroalkyl, C₁₋₅haloalkyl, and C₃₋₆cycloalkyl;
each R¹¹ and R¹² is independently selected from H, C₁₋₅alkyl, C₂₋₅alkenyl, C₂-₅alkynyl, C₁₋ sheteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂-₅alkynyl, C₁₋ ₅heteroalkyl, C₁₋₅haloalkyl, and C₃₋₆cycloalkyl.

In some embodiments, the pharmaceutical composition is administered parenterally. In some embodiments, the pharmaceutical composition is administered subcutaneously. In some embodiments, the pharmaceutical composition is subcutaneously injected directly into an angiolipoma. In some embodiments, the pharmaceutical composition is subcutaneously injected directly into an angiolipoma at a dosage of from about 0.05 to about 0.1 mL per angiolipoma. In some embodiments, the pharmaceutical composition is subcutaneously injected directly into an angiolipoma at a dosage of from about 0.1 to about 0.4 mL per angiolipoma. In some embodiments, the pharmaceutical composition is subcutaneously injected directly into an angiolipoma at a dosage of from about 0.4 to about 1 mL per angiolipoma. In some embodiments, the pharmaceutical composition is subcutaneously injected directly into an angiolipoma at a dosage of from about 1 to about 2 mL per angiolipoma. In some embodiments, the pharmaceutical composition is subcutaneously injected directly into an angiolipoma at a dosage of less than about 0.05 mL per angiolipoma. In some embodiments, the pharmaceutical composition is subcutaneously injected directly into an angiolipoma at a dosage of about 0.05 mL per angiolipoma. In some embodiments, the pharmaceutical composition is subcutaneously injected directly into an angiolipoma at a dosage of about 0.1 mL per angiolipoma. In some embodiments, the pharmaceutical composition is subcutaneously injected directly into an angiolipoma at a dosage of about 0.2 mL per angiolipoma. In some embodiments, the pharmaceutical composition is subcutaneously injected directly into an angiolipoma at a dosage of about 0.3 mL per angiolipoma. In some embodiments, the pharmaceutical composition is subcutaneously injected directly into an angiolipoma at a dosage of about 0.4 mL per angiolipoma. In some embodiments, the pharmaceutical composition is subcutaneously injected directly into an angiolipoma at a dosage of about 0.5 mL per angiolipoma. In some embodiments, the pharmaceutical composition is subcutaneously injected directly into an angiolipoma at a dosage of about 0.6 mL per angiolipoma. In some embodiments, the pharmaceutical composition is subcutaneously injected directly into an angiolipoma at a dosage of about 0.7 mL per angiolipoma. In some embodiments, the pharmaceutical composition is subcutaneously injected directly into an angiolipoma at a dosage of about 0.8 mL per angiolipoma. In some embodiments, the pharmaceutical composition is subcutaneously injected directly into an angiolipoma at a dosage of about 0.9 mL per angiolipoma. In some embodiments, the pharmaceutical composition is subcutaneously injected directly into an angiolipoma at a dosage of about 1.0 mL per angiolipoma. In some embodiments, the pharmaceutical composition is subcutaneously injected directly into an angiolipoma at a dosage of about 1.1 mL per angiolipoma. In some embodiments, the pharmaceutical composition is subcutaneously injected directly into an angiolipoma at a dosage of about 1.2 mL per angiolipoma. In some embodiments, the pharmaceutical composition is subcutaneously injected directly into an angiolipoma at a dosage of about 1.3 mL per angiolipoma. In some embodiments, the pharmaceutical composition is subcutaneously injected directly into an angiolipoma at a dosage of about 1.4 mL per angiolipoma. In some embodiments, the pharmaceutical composition is subcutaneously injected directly into an angiolipoma at a dosage of about 1.5 mL per angiolipoma. In some embodiments, the pharmaceutical composition is subcutaneously injected directly into an angiolipoma at a dosage of about 1.6 mL per angiolipoma. In some embodiments, the pharmaceutical composition is subcutaneously injected directly into an angiolipoma at a dosage of about 1.7 mL per angiolipoma. In some embodiments, the pharmaceutical composition is subcutaneously injected directly into an angiolipoma at a dosage of about 1.8 mL per angiolipoma. In some embodiments, the pharmaceutical composition is subcutaneously injected directly into an angiolipoma at a dosage of about 1.9 mL per angiolipoma. In some embodiments, the pharmaceutical composition is subcutaneously injected directly into an angiolipoma at a dosage of about 2.0 mL per angiolipoma. In some embodiments, the pharmaceutical composition is subcutaneously injected directly into an angiolipoma at a dosage of greater than about 2.0 mL per angiolipoma.

### EXAMPLES

### Example 1 Clinical Trial

The clinical trial described here was an open label, phase 2a clinical trial for the evaluation of safety and efficacy of Compound 1 for the treatment of women and men with nodular Dercum’s disease (DD). Six (6) women or men with DD were given a single treatment session in which Compound 1 was injected into several lipomas per patient. Prior to injection, angiolipomas (i.e. lipomas containing a fibrous capsule, 11 lipomas in total) were defined and the height of each lipoma was evaluated by ultrasound (US). Individual lipoma pain score was also determined prior to injection using the comparative pain scale.

DD patients were followed up on days 28 and 56 post-injection to determine lipoma height and lipoma pain. Tables 1 and 2 represent assessments of lipoma height and lipoma pain before and after Compound 1 dosing. Angiolipoma size and height were assessed by US. Compound 1 injection into angiolipomas significantly reduced their height and alleviated pain.

TABLE 1

Relative change vs. baseline in angiolipoma height (* P<0.05 vs baseline)

| Lipomas Height | | N of Lipomas | Mean height (mm) | Std | Relative change in lipoma height (%) | P-value vs baseline |
|---|---|---|---|---|---|---|
| Baseline | Fibrotic tissue associated with the capsule | 11 | 10.5 | 3.6 | | |
| Day 28 | Fibrotic tissue associated with the capsule | 11 | 8.8 | 3.0 | -16.2 | 0.24 |
| Day 56 | Fibrotic tissue associated with the capsule | 11 | 7.4 | 3.1 | -26.2 | 0.043* |

TABLE 2

Relative change vs. baseline in angiolipoma pain (* P<0.05 vs baseline)

| Lipomas pain | | N of Lipomas | Mean pain score | Std | Relative change in lipoma pain (%) | P-value vs baseline |
|---|---|---|---|---|---|---|
| Baseline | Fibrotic tissue associated with the capsule | 11 | 5.2 | 1.7 | | |
| Day 28 | Fibrotic tissue associated with the capsule | 11 | 3 | 2.9 | -42.3 | 0.045 |
| Day 56 | Fibrotic tissue associated with the capsule | 11 | 2.7 | 2.7 | -48.1 | 0.019* |

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

What is claimed is:

1. A pharmaceutical composition comprising a compound of Formula (I): 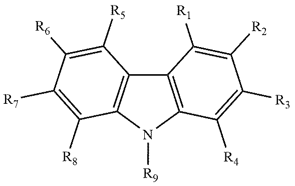
or a pharmaceutically acceptable salt thereof, wherein:
each of R¹, R², R³, R⁴, R⁵, R⁶, R⁷, and R⁸ is independently selected from H, halogen -CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁₋ sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋ 5alkynyl, C₁₋₅heteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁₋ sheteroalkyl, C₁₋₅haloalkyl, and C₃₋₆cycloalkyl;
for use in the treatment of angiolipoma and any symptoms or conditions associated therewith.

2. The pharmaceutical composition of claim 1, wherein R₉ is C₁-C₉ alkyl substituted with at least one quaternary amino group.

3. The pharmaceutical composition of claim 1, wherein the at least one ammonium group is of Formula (V): 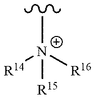
wherein each of R¹⁴, R¹⁵, and R¹⁶ is independently selected from C₁₋₉alkyl, C₂₋₉alkenyl, and C₂₋₉alkynyl.

4. The pharmaceutical composition of claim 3, wherein each of R¹⁴, R¹⁵, and R¹⁶ is independently C₁₋₉alkyl.

5. The pharmaceutical composition of any one of the preceding claims, wherein at least one of R¹, R², R³, and R⁴ is halogen.

6. The pharmaceutical composition of any one of the preceding claims, wherein at least one of R⁵, R⁶, R⁷, and R⁸ is halogen.

7. The pharmaceutical composition of any one of the preceding claims, wherein at least one of R¹, R², R³, and R⁴ is halogen and at least one of R⁵, R⁶, R⁷, and R⁸ is halogen.

8. The pharmaceutical composition of any one of claims 5 to 7, wherein the halogen is bromo.

9. The pharmaceutical composition of any one of the preceding claims, wherein at least one of R¹, R², R³, and R⁴ is OH.

10. The pharmaceutical composition of any one of the preceding claims, wherein at least one of R⁵, R⁶, R⁷, and R⁸ is OH.

11. The pharmaceutical composition of any one of the preceding claims, wherein at least one of R¹, R², R³, and R⁴ is nitro and at least one of R⁵, R⁶, R⁷, and R⁸ is nitro.

12. The pharmaceutical composition of any one of the preceding claims, wherein the compound of Formula (I) is selected from:
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, and
5-(3,6-dibromo-9H-carbazol-9-yl)-N,N,N-trimethylpentan-1-aminium.

13. The pharmaceutical composition of any one of the preceding claims, wherein the pharmaceutical composition comprises less than about 50% water by weight.

14. The pharmaceutical composition of any one of the preceding claims, wherein the pharmaceutical composition comprises less than about 30% water by weight.

15. The pharmaceutical composition of any one of the preceding claims, wherein the pharmaceutical composition comprises less than about 10% water by weight.

16. The pharmaceutical composition of any one of the preceding claims, wherein the pharmaceutical composition comprises from about 0% to about 30% water by weight.

17. The pharmaceutical composition of any one of the preceding claims, wherein the pharmaceutical composition comprises at least about 0.1% by weight of the compound of Formula (I).

18. The pharmaceutical composition of any one of the preceding claims, wherein the pharmaceutical composition comprises from about 0.1% to about 10% by weight of the compound of Formula (I).

19. The pharmaceutical composition of any one of the preceding claims, wherein the pharmaceutical composition comprises from about 1% to about 5% by weight of the compound of Formula (I).

20. The pharmaceutical composition of any one of the preceding claims, wherein the pharmaceutical composition further comprises at least one additional active agent.

21. The pharmaceutical composition of any one of the preceding claims, wherein the pharmaceutical composition is formulated for parenteral administration.

22. The pharmaceutical composition of claim 21, wherein the pharmaceutical composition is formulated for subcutaneous injection directly into an angiolipoma.

23. A pharmaceutical composition according to claim 21, wherein the pharmaceutical composition is formulated for subcutaneous injection directly into an angiolipoma at a dosage of from about 0.05 to about 0.1 mL per angiolipoma.

24. A pharmaceutical composition according to claim 21, wherein the pharmaceutical composition is formulated for subcutaneous injection directly into an angiolipoma at a dosage of from about 0.1 to about 0.4 mL per angiolipoma.

25. A pharmaceutical composition according to claim 21, wherein the pharmaceutical composition is formulated for subcutaneous injection directly into an angiolipoma at a dosage of from about 0.4 to about 1 mL per angiolipoma.

26. A pharmaceutical composition according to claim 21, wherein the pharmaceutical composition is formulated for subcutaneous injection directly into an angiolipoma at a dosage of from about 1 to about 2mL per angiolipoma.

27. A kit comprising the pharmaceutical composition of any one of claims 1 to 26, means for administration of the pharmaceutical composition, and instructions for use thereof.

28. The kit of claim 27, wherein the kit further comprises at least one additional therapeutic agent.

29. A method of treating angiolipoma in a subject in need thereof, comprising administering to the subject a pharmaceutical composition comprising a compound of Formula (I): 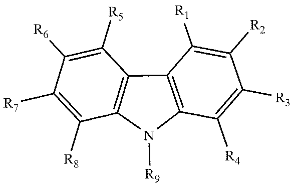
or a pharmaceutically acceptable salt thereof, wherein:
each of R¹, R², R³, R⁴, R⁵, R⁶, R⁷, and R⁸ is independently selected from H, halogen, —CN, —NO₂, —OR¹⁰, —SR¹⁰, —S(═O)R¹⁰, —S(═O)₂R¹⁰, -NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is selected from C₁₋₉alkyl, C₂₋₉alkenyl, C₂₋₉alkynyl, and 3- to 10-membered heterocycloalkyl; wherein R⁹ is substituted with at least one quaternary amino group or phosphonium group;
each R¹⁰ is independently selected from H, C₁₋₅alkyl, C₂₋₅alkenyl, C₂₋₅alkynyl, C₁₋ sheteroalkyl, C₁₋₅haloalkyl, and C₃₋₆cycloalkyl;
each R¹¹ and R¹² is independently selected from H, C₁₋₅alkyl, C₂₋₅alkenyl, C₂₋ salkynyl, C₁₋₅heteroalkyl, C₁₋₅haloalkyl, C₃₋₆cycloalkyl; or an R¹² and an R¹³ may be taken together along with the nitrogen atom to which they are attached to form a 3- to 10-membered heterocycloalkyl; and
each R¹³ is independently selected from H, C₁₋₅alkyl, C₂₋₅alkenyl, C₂₋₅alkynyl, C₁₋ sheteroalkyl, C₁₋₅haloalkyl, and C₃₋₆cycloalkyl.

30. The method of claim 29, wherein the pharmaceutical composition is administered parenterally.

31. The method of claim 29 or 30, wherein the pharmaceutical composition is administered subcutaneously.

32. The method of any one of claims 29 to 31, wherein the pharmaceutical composition is subcutaneously injected directly into an angiolipoma.

33. The method of any one of claims 29 to 32, wherein the pharmaceutical composition is subcutaneously injected directly into an angiolipoma at a dosage of from about 0.05 to about 0.1 mL per angiolipoma.

34. The method of any one of claims 29 to 32, wherein the pharmaceutical composition is subcutaneously injected directly into an angiolipoma at a dosage of from about 0.1 to about 0.4 mL per angiolipoma.

35. The method of any one of claims 29 to 32, wherein the pharmaceutical composition is subcutaneously injected directly into an angiolipoma at a dosage of from about 0.4 to about 1 mL per angiolipoma.

36. The method of any one of claims 29 to 32, wherein the pharmaceutical composition is subcutaneously injected directly into an angiolipoma at a dosage of from about 1 to about 2mL per angiolipoma.
